(19) United States
(12) Patent Application Publication
(10) Pub. No.: US 2004/0023913 A1
(43) Pub. Date: Feb. 5, 2004

(54) Agent with inactivates pathogens, comprising an element that bonds with nucleic acids and the use thereof

(76) Inventors: Hans-Juergen Neumann, St. Wendel (DE); Helmut Knoller, Ulm (DE)

Correspondence Address: JACOBSON HOLMAN PLLC, 400 SEVENTH STREET N.W., SUITE 600, WASHINGTON, DC, 20004, US

(21) Appl. No.: 10/399,048

(22) PCT Filed: May 4, 2001
(86) PCT No.: PCT/EP01/05034

(51) Int. Cl. A61K 48/00; C07H 21/02

(52) U.S. Cl. 514/44; 536/23.1; 536/17.1

(57) ABSTRACT

The invention relates to a pathogen-inactivating agent, as well as its use, whereby the agent contains an element that binds to nucleic acids of the pathogens, and a conjungate that destroys nucleic acid. The conjungate is created from a metal chelate complex, in which the metal can change between at least two oxidation levels. In particular, the agent can be used in physiological liquids, such as blood, or blood fractions for the inactivation of viruses.

The invention relates to an agent for the inactivation of pathogenic germs, such as viruses and bacteria, and its use, preferably in physiological solutions, such as blood, or blood fractions. In particular, the pathogen-inactivating agent consists of an element binding to nucleic acids, and a conjungate destroying the effect of nucleic acid, as well as an optional spacer located in-between, whereby the conjungate is a metal chelate complex.

With an increasing spread of pathogenic germs, it becomes more difficult to find suitable means for the inactivation of germs. The problem is intensified by the fact that viruses especially have a high degree of variability, which makes previously used antiviral preparations ineffective. It should be possible to create a pathogen-inactivating agent that acts very specifically on one hand in order to specifically destroy a certain type of virus, but to also create the same non-specifically on the other hand, so that a large amount of pathogenic germs are inactivated.

For this purpose it seems useful, as has already been applied in broad ranges of prior art, to select its nucleic acids as the pathogenic germ's target, i.e., the DNA (desoxiribonucleic acid), or RNA (ribonucleic acid) of the pathogenic germ. In the case of a specific attachment of the pathogen-inactivating agent, a very selective inhibition can be achieved, as it is shown in Nucleic Acid Research, Vol. 15 No. 23(1), pages 9909 to 9919 by the authors Zerial et al., in which they describe the selective inhibition of Type A influenza viruses by means of a highly specific oligonucleotide with a reactive conjungate. This is selectivity based on the fact that the oligonucleotide to be used has been synthesized specifically as a complementary strand to a conservative DNA sequence. As is generally known, the bases cytosine always pair with guanine and thymine, or their counter-part of the RNA, and uracil always with adenine. These adhesion forces depend on the temperature on one hand, and are dependent on the quantity of the matching pairs, on the other hand. Therefore, this method can be used to specifically inhibit a type of virus, or a class of viruses with common properties. WO 95/32986, and WO 92/05284 describe antisense oligonucleotides, which possess the pathogenic germ inhibiting properties. The oligonucleotide consists of sequences, each of which are also complementary to the nucleic acid sequence of the pathogenic germ, or in case of WO 92/05284, bind to specific strategic sides of the microbes. Consequently, only specific pathogenic target germs are included, and the oligonucleotides are therefore not suitable for various pathogenic germs, particularly not for all types of viruses.

In order to also destroy viruses, agents are often used, which have highly oxidizing or alkylizing effects, and/or attach to the genome. Therefore, these agents are mutagenic or cancerous also for other cells. Furthermore, the problem exists that they still fail in the case of a large amount of viruses due to their heterogeneity. Although an attachment to nucleic acids should occur in order to also activate viruses, this should be as non-specific as possible.

In the case of a non-specific attachment, there are 4 groups of attachment reagents that have been described in U.S. Pat. No. 5,691,132, the intercalators, the so called “groove binders,” whereby they are separated into “minor groove binders,” and “major groove binders.” Intercalators are agents that position themselves between the two nucleic acid strands of the DNA. A DNA double helix has larger grooves in a spiral, and smaller ones, which can attach themselves to the “major,” or “minor groove binders.” Furthermore, as also stated in detail in the application establishing priority DE 100 51 628.9, the agents are those that bind to the phosphate group framework of nucleic acids, as well as non-specified oligonucleotides.

With the use of blood products in particular, such as with the transfusion of human blood, or with the separation of proteins from blood, or from blood fractions, a large demand exists of destroying potential pathogenic germs before the products are circulated to their intended use. According to prior art, diverse methods and agents are already known, which cause the inactivation of pathogenic germs. They all have in common that they either have cumbersome side effects, or require extensive handling.

U.S. Pat. No. 5,691,132 claims a method in which pathogens are inactivated in a blood product, whereby an intercalator is added to the blood product, which can already cause the destruction of the DNA by means of its non-specified attachment to the DNA of the pathogens. However, this intercalator is able to also attack other DNA, and therefore has a mutagenic, or cancerous effect. It must therefore be removed from the blood product after the completed inactivation by means of an adsorbent agent, which requires an additional handling step that is not without risk for the person performing this task.

WO 00/04930 also shows a method and a device for the inactivation of microorganisms, whereby the attachment to the nucleic acid of the microorganisms is to occur by means of a non-toxic agent, which is initially activated by radiation, and which then destroys the DNA. Such agents are alloxacines, or isoalloxacines, which riboflavin or vitamin B2 are also a part of. Although riboflavin should belong to the intercalators due to its benzenoid multiple ring structure, it does not seem to have any mutagenic or cancerous properties. Not until the irradiation with light does riboflavin have the pathogen-activating effect. The disadvantage of this method is the radiation, which represents an additional processing step, and also has a damaging effect on other cells. Additionally, undesired byproducts are created, for instance, riboflavin can be transmuted to lumiflavin, or lumichromium.

It is also known to induce strand breaks in the DNA or RNA by means of a conjungate with a reactive group, such as an EDTA iron chelating complex (EDTA=ethylene Diamine Tetra Acetate). In the literature of Genes 72 (1988), pages 361-371, the authors Boidet-Foget et al. show that a specific transecting of single-strand and double-strand DNA is possible. Also, Biochemistry Vol. 29 No. 6 (Feb. 13, 1990) of Celander and Chech has shown that the iron (II)-EDTA catalyzed transecting of RNA and DNA has no, or just a slight specificity.

This property therefore has been known in molecular biology for years, and is used for so-called “mapping.” In this case, a protein mostly binds to a specific location of the DNA, whereby this location, which is protected by the protein, cannot be attached by Fe(II) complexes. This makes it possible to identify attachment locations to a gene. Examples for such mapping can be found in Biochemistry (1994), Vol. 33, pages 9831 to 9844, or Biochemistry Vol. 35 No. 37 (Sept. 7, 1996) page 11931 and following pages.

It is the task of the invention at hand to provide an agent for the inactivation of viruses, which does not contain the disadvantages of the pathogen-inactivating agent that has been known from prior art, for example, which does not require any extensive handling. The task is solved by means of the characteristics of the first claim.

Characteristic for the invention is the functional separation of the pathogen-inactivating agent into two areas, whereby one area is responsible for the attachment to nucleic acids, and the second area is responsible for the destruction of the nucleic acids. The attachment can occur either specifically, or non-specifically, however, it is preferably non-specific, because all pathogenic germs should be reached, if possible.

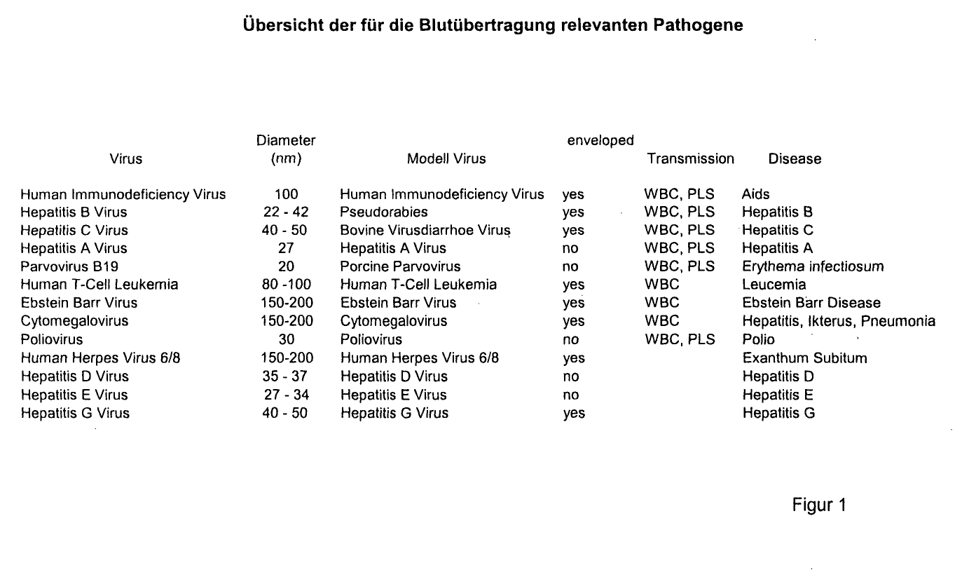

In this regard, pathogenic germs are understood as all germs with a genome leading to disease patterns for organisms, such as bacteria, or viruses. FIG. 1 shows a table of the most important viruses and bacteria relevant in a blood transfusion. It shows that some viruses have double-stranded DNA, others have single-stranded RNA, and viruses can be coated, or uncoated.

While intercalators, phosphate bridge binders, or “groove binders” non-specifically bind to DNA per se, the oligonucleotides provide the opportunity to adjust the desired specificity by means of complementary base pair formation. In this case, the specificity becomes smaller, the smaller the oligonucleotide is. A low specificity is achieved by means of the length of an oligonucleotide of a maximum of 6 specific nucleotides, whereby the term specific nucleotide means that its base is selected from the group of the specific bases guanine, adenine, thymine, uracil, and/or cytosine. Statistically, such a synthetic oligonucleotide finds several hits on the genome so that all pathogenic germs can be inactivated, if possible, especially all virus types, if possible. If the nucleotide succession of specific nucleotides is longer than 6 nucleotides, the hits on the genome become significantly smaller, which causes the specificity, and as a result thereof also the selectivity to increase.

In addition to the 5 specific bases named above, so-called universal bases also exist, which form a base pair formation with all, or at least with several bases. These can be synthetic—as is the 5-nitroindol—or natural, as inosine. Inosine, which is a base of the transfer RNA (tRNA), can form a base pair with cytosine, adenosine, and uracil. With the use of the inactivation agent in blood, or blood products that are intended for a transfusion, inosine is accordingly preferred as a universal base. However, other universal bases are also possible for in vitro use.

The use of a universal base enables a bond of the oligonucleotide at higher temperatures, whereby the advantage remains, however, to achieve as many bonds with genomes of pathogenic germs, as possible, with only few specific nucleotides. The length of the genome is also a decisive factor of the frequency of an attachment.

An oligonucleotide is also capable of binding a double-stranded DNA (dsDNA), and to form a so-called triple helix with it. It has also been shown that a succession of equal nucleotides bind to the dsDNA particularly well with the bases thymine, or cytosine. For this reason, such a structure is preferred, especially preferred is the succession of at least three cytosines, as cytosine has about twice the binding strength to DNA, than thymine.

Furthermore, one side of the oligonucleotide is advantageously equipped with a protective group, which at least delays the degradation of the oligonucleotide. Again, for the purpose of in vitro use, an additional nucleotide is used, as it is natural and does not have a toxic effect in humans and animals. In order to develop its potency as a protective group, however, the additional nucleotide is incorporated symmetrically reversed to the other nucleotides so that the RNA s or DNA s, i.e., the nucleotide-degrading enzymes are provided with as little chance for attack, as possible. This protective group nucleotide does not contribute to a bond on a complementary strand, and is therefore not counted in the length determination of an oligonucleotide.

Examples for agents that bind to the phosphate group framework are spermines, spermidines, and other polyamines, aflatoxins, for instance, are the “major groove binders,” and among others, the triarylemethane dyes of the Hoechst 33258, and other Hoechst dyes belong to the “minor groove binders,” berenile, DAPI (4′, 6′diamidine-2phenylindole), distamycin, mitomycin, netropsine, and other lexitropsines.

The largest group of substances binding to DNA, however, is the intercalators. These are usually flat aromatic systems that form a bridge between the double strands by means of their π-electron system, instead of the usually existing hydrogen bridges. These aromatic compounds can be heterocyclic or benzenoid, mostly however, intercalators consist of multi-ring systems.

The following are representatives of the group: Acridine, acridone, proflavin, acriflavine, aloxazine, isoalloxazine, porphine, or porphyrine, actinomycin, anthracyclinon, beta-rhodomycin A, daunamycin, thiaxanthenone, miracil D. anthramycin, mitomycin, echinomycin, quinomycin, triostine, diacridine, ellipticene, (also dimere, trimere, and analoga), norphilline A, fluorene, and fluorenone, fluorenodiamine, quinacrin, benzoacridine, phenazine, phenanthradine, phenothiazine, chlorpromazine, phenoxazine, benzothiazole, xanthene, and thioxanthene, anthraquinone, anthrapyrazoles, benzothiopyranoindole, 3.4-benzopyrene, benzopyrenediolepoxide, 1-phenyloxirane, benzanethracen-5.6-oxide, benzodipyrone, quinolone, chloroquine, quinine, phenylquinolinecarboxamide, furocoumarine, as well as psorale, and isopsorale, ethidium salts, propidium, and coralyne, ellipticin-cations, and their derivatives, polycyclic hydrocarbons, and their oxirane derivatives, as well as echinomycin.

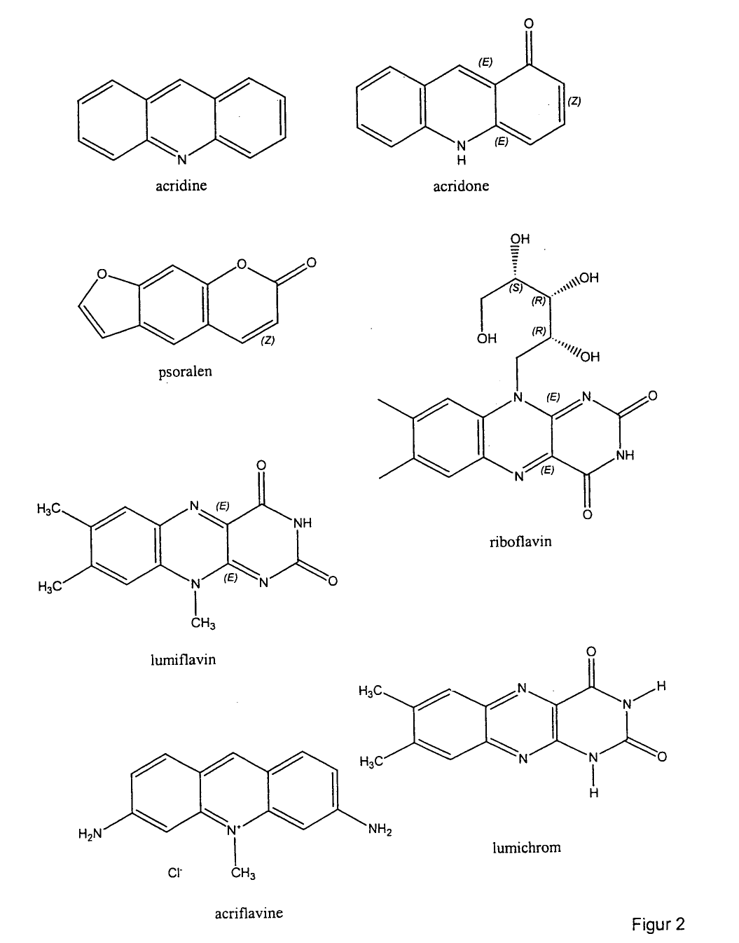

According to the invention, acridines, a triple 6-ring system, and their derivatives, such as alloxazines and isoalloxazines, as well as porphines, or substituted porphines, such as porphyrines and their derivatives, are preferred. On the one hand, these agents are selected because of their strong binding capability, and on the other, because non-toxic, non-mutagenous, and non-cancerous acting derivatives can also be selected from this group for use in biological systems. Heme, for example, a component of blood, consists of a porphyrine ring that is populated by Fe(II), and created of 4 indoles. Vitamin B2, or even riboflavin can also be found among the alloxazines, which is especially preferred as the nucleic acid binding element. FIG. 2 shows suitable multiple-ring systems.

The actual destruction of the genome occurs by means of the reactive conjungate that is attached to the free end of the oligonucleotide, which is a metal chelate complex, whereby the metal can change between two oxidation levels. The pathogen-inhibiting effect is probably initiated by transecting the nucleic acids according to the Fenton mechanism, and is thereby based on the reducing properties of the metal. Accordingly, the metal itself is oxidized, and can be rejuvenated by using a reducing agent.

According to the invention, all complexes that cause a destruction of the genome of pathogens can be used. Complexes consist of a central atom, or according to the invention, of a central ion as well as ligands. For the most part, heavy metal cations of a high charge with small ion radii usually act as the central ions. The ligands are often anions, however, uncharged molecules often also occur as ligands. When speaking of compounds that possess two or more functional groups, i.e., that can populate several coordination points of the central ion, they are called bi-, or multidental ligands, such as ethylendiamine, or exalate. The charge of the complex corresponds to the sum of the charge of the single ions forming it.

The presence of two or more coordination points in one organic molecule leads to a ring formation. It preferably occurs whenever a tension-free 5-, or 6-ring can form. Generally, compounds in which a molecule is closed in a ring via a metal ion by means of coordination, are called chelating complexes, or chelate. Uncharged chelating complexes are called internal chelating complexes; the charges of the central ion, and those of the ring-forming ligands offset each other, such as with nickel-diacethyldioxime, or magnesium-oxinate, in order to name a few examples of known complexes.

The so-called complexions, the most important representative of which are EDTA and nitriletriacetic acid, are chelating agents that partially form very stable, water-soluble complexes with almost all cations, including natural alkali. Another example is EDPA iron (diethyleneamine pentaacetate).

According to the invention, an iron(II) EDTA chelating complex, or an iron(III) EDTA chelating complex is preferred. This complex is especially suitable for use in biological systems, such as for the inactivation of viruses in blood, or blood fractions, because both iron, as well as EDTA are bio-compatible, and already approved medication can be used without any risk for the promotion of the human hemogram, and therefore for the infusion into the human or animal body. Particularly advantageous for such a use is the combination with an element that is also biocompatible, such as the said riboflavin. A spacer or linker that is also biocompatible therefore renders itself useful with the use of a spacer between the iron EDTA complex and riboflavin.

For this purpose, iron can exist in the oxidation levels 2+or 3+. If iron exists in the oxidation level 2+, each iron molecule can develop its effect on one hand, and is oxidized into iron (III) itself during this process. By reducing the iron (III) again to iron (II) by the use of an additional reducing agent, the effect of the complex can be repeated indefinitely so that only small amounts of the pathogen-inactivating agent need to be used. Also, a harmless, biocompatible agent will be used in vivo as the reducing agent, such as vitamin C or ascorbic acid, or its salt ascorbate. Furthermore, the use of Fe(III) is generally possible so that the inactivation reaction can start via the addition of the reduction agent according to the invention.

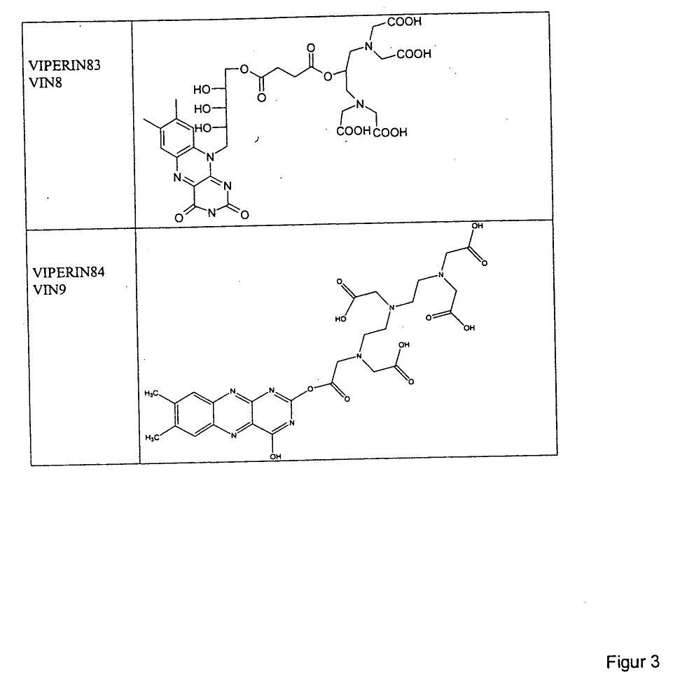

The combination of an element binding to nucleic acid according to the invention, as well as possibly a spacer, and the conjungate destroying the nucleic acid will be hereinafter referred to as VIPERIN. FIG. 3 shows various VIPERINS, whereby VIPERIN81, or VIN6, shows the above preferred design of a compound of riboflavin and iron EDTA. All tests shown below have been performed using VIPERIN 1.

The above mentioned virus activation is illustrated in FIG. 4 in dependency of the sodium ascorbate concentration:

A phage titer of an MS2 phage of 1×10⁹ lbs/ml of water was incubated for 4 hours at room temperature with 50 μg/ml of VIPERIN1. The acridine base body is suitable to bind to the nucleic acids of the phage MS2, while the attached iron EDTA complex initiates the destruction of the nucleic acid. VIPERIN1 is charged with iron(III) (not shown in FIG. 3). Beginning with a sodium ascorbate concentration of 100 μmol/l the virus activation (VI), applied in the 10^{th} logarithm, increases steadily. The higher the ascorbate concentration, the more iron (III) is reduced to iron(II), the “active” form of the EDTA iron complex, and the conjungate destroying nucleic acid is therefore more active. Interestingly, the concentration of ascorbate in the human body is about 100 μM so that it can be assumed that no, or merely a slight activation of the iron(III) EDTA complex occurs in blood, or in blood fractions.

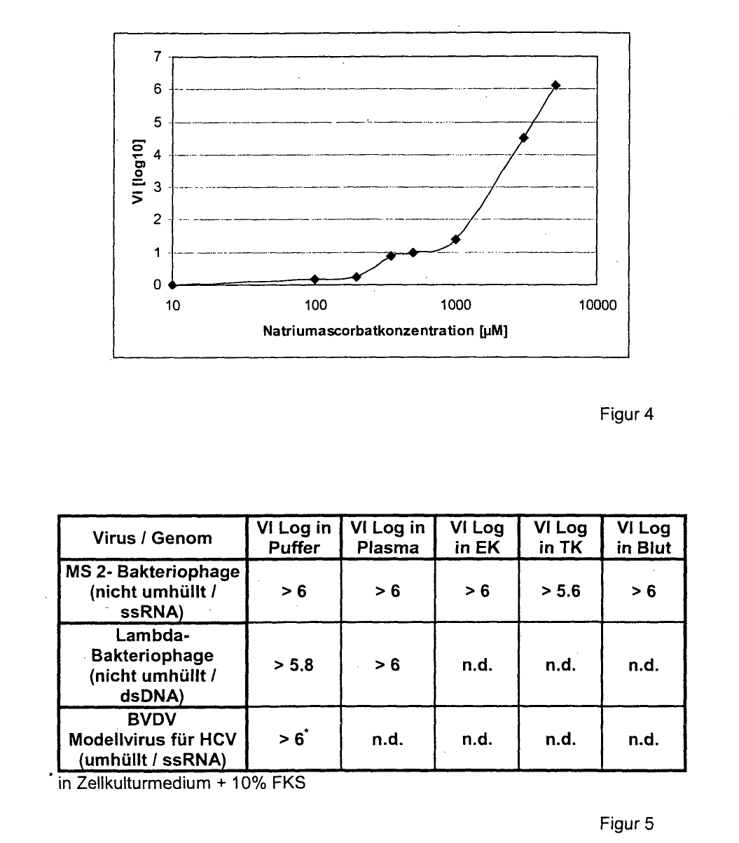

In order to substantiate this thesis, the MS2 bacteriophage was exposed to various media as a non-sheathed virus with single-stranded RNA (ssRNA), also to physiological media, such as blood, or blood fractions. Furthermore, VIPERIN1's capability of inactivating was detected in additional viruses. In order to ensure a heterogeneity that is as high as possible, the Lamda bacteriophage was also incubated as a non-sheathed virus, however with dsDNA with VIPERIN1, as well as in tris-buffer pH 7.5, as well as in blood plasma, as well as the BVDV, a model virus for sheathed viruses also with single-stranded RNA in buffer. FIG. 5 illustrates the results in a table. The virus inactivation is stated in the 10^{th} logarithm, whereby log 6 illustrated the detection limit of the tests. Each sterile tris-buffer was prepared with 6 g of tris, 2 g of MgSO₄*7H₂O, 5.8 g of NaCl on 1 l of water, as well as the addition of 5 ml of 20% albumin solution. 50 μg/ml of VIPERIN each was charged with iron(III) for 4 hours at room temperature, each with 1×10⁹ lbs of phage titer per ml of water and 5 mM of sodium ascorbate. The MS2 phage was reduced by at least 6 logarithm levels in buffer, plasma, erythrocyte concentrate, and in whole blood, in thrombocyte concentrate by more than 5.6 levels. The Lamda phage was reduced in buffer by more than 5.6, and in plasma by more than 6 logarithm levels, the BVDV in buffer by more than 6.

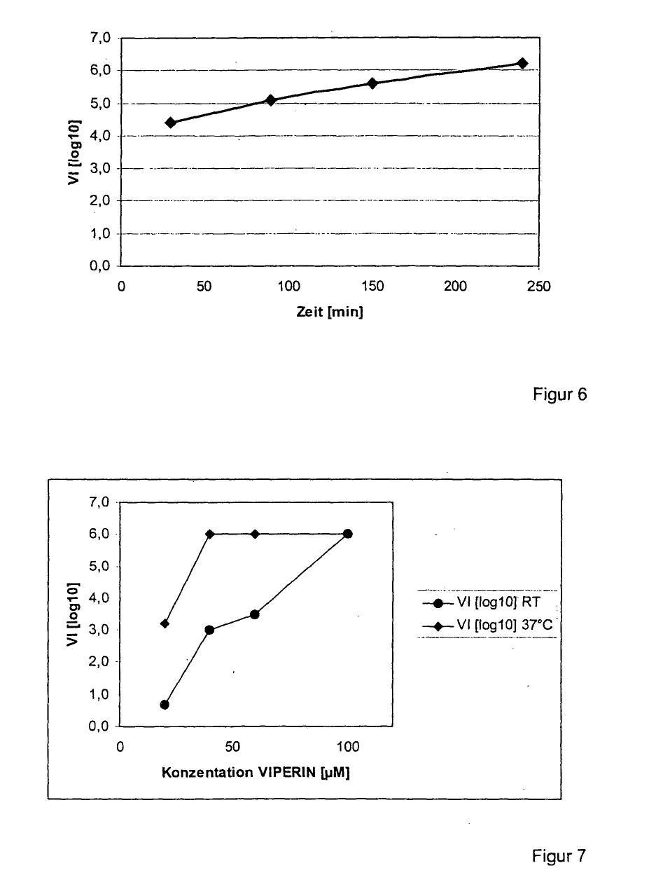

Furthermore, the potency of VIPERIN was examined as an example by means of inactivation of the phage MS2 in dependency of time, temperature, or concentration. The incubation preparation each consisted of a virus charge of 10⁹ lbs/ml, VIPERIN charged with iron(III), and 5 mM of sodium ascorbate. FIG. 6 graphically illustrates the result that the inactivation of MS2 increases by almost 2 logarithm levels over a time period of about 4 hours; the inactivation is therefore time-dependent.

FIG. 7 graphically shows the result of the virus inactivation once at room temperature (RT), and incubated at 37° C. for 4 hours. For one, the virus inactivation is temperature-dependent. It was found that with low VIPERIN concentrations, the inactivation is higher at 37° C., but shows its maximum value of virus activity reduction of at least 6 logarithm levels already at about 40 μM of VIPERIN. On the other hand, the virus inactivation is concentration-dependent. With incubation at room temperature, the inactivation of the MS2 phage steadily increases with increasing concentrations of VIPERIN. Although the maximum measurable value of inactivation of 6 logarithm levels with an incubation at 37° C. in MS2 is already achieved at 40 μM of VIPERIN, an increase of the inactivation capability of 3 logarithm levels can also be detected starting at only 20 μM of VIPERIN.

1. Pathogenic inactivating agent with an element binding to nucleic acids, characterized in that the element binding to nucleic acid contains a conjungate destroying nucleic acid, as well as possibly a spacer between both parts, and that the conjungate is a metal chelate complex, in which the metal can be oxidized, or reduced at various oxidation levels.

2. Pathogen-inactivating agent according to claim 1, characterized in that the element binding nucleic acid is selected from the group of oligonucleotides, the intercalators, the phosphate group framework binder, and/or the “groove binders.”

3. Pathogen-inactivating agent according to claim 1, characterized in that the metal chelate complex is selected from the group of the internal complexes, or from the group of complexions.

4. Pathogen-inactivating agent according to claims 1 and 3, characterized in that the chelating agent is selected from the group of dioximes, oxinates, EDTA complexes, DTPA complexes, EDTA complex derivatives, DTPA complex derivatives, nitriletriacetic acid, and/or porphines.

5. Pathogen-inactivating agent according to claims 1 and 3, characterized in that the metal of the metal chelate complex is selected from the 8^{th} secondary group of the periodic table of the elements.

6. Pathogen-inactivating agent according to claims 1 and 3, characterized in that the metal of the metal chelate complex is iron at the oxidation level II.

7. Pathogen-inactivating agent according to claims 1 and 3, characterized in that the metal of the metal chelate complex is iron at the oxidation level III.

8. Pathogen-inactivating agent according to one or more of the previous claims, characterized in that the metal chelate complex is iron(III) EDTA.

9. Pathogen-inactivating agent according to one or more of the previous claims, characterized in that a reduction agent is additionally contained.

10. Pathogen-inactivating agent according to claim 9, characterized in that the reduction agent is ascorbic acid, or one of its salts.

11. Pathogen-inactivating agent according to one or more of the previous claims, characterized in that the ascorbic acid or its salts exists at a concentration of more than 100 μmol/l.

12. Pathogen-inactivating agent according to claims 1 and 2, characterized in that the oligonucleotide is single-stranded, and has less than 7 specific nucleotides from the group thymine, adenine, cytosine, guanine, and/or uracil.

13. Pathogen-inactivating agent according to claim 12, characterized in that the oligonucleotide has additional universal bases selected from the group inosine, and/or 5-notroindol.

14. Pathogen-inactivating agent according to claims 1 and 2, characterized in that the “groove binders” are selected from the group of “minor groove binders,” containing distamycine, mitomycine, netropsin, lexitropsines, berenile, indoles, and/or triarylmethane dyes, and of the group “major groove binders,” containing aflatxoines.

15. Pathogen-inactivating agent according to claims 1 and 2, characterized in that phosphate group framework binders are selected from the group of spermines, spermidines, and other polyamines.

16. Pathogen-inactivating agent according to claims 1 and 2, characterized in that the intercalators are selected from the group of benzenoid aromatic compounds, and/or of non-benzenoid aromatic compounds, such as heteroaromatic compounds.

17. Pathogen-inactivating agent according to claim 16, characterized in that the aromatic compounds consist of multiple ring systems.

18. Pathogen-inactivating agent according to claims 16 and 17, characterized in that the aromatic compounds are acridine, acridone, alloxacin, or isoaloxacin, such as riboflavin and other flavins, porphines, or porphyrines, such as heme, mycine, fluorine, acridine, psoralene, ethidium salts, oxirane, coumarone, psoralene, phenyl compounds, xanthene, ellipticene, quinolone, chloroquine, quinine, propidium coralyne, and/or their derivatives.

19. Pathogen-inactivating agent according to one or more of the previous claims, characterized in that riboflavin is preferred as the intercalator.

20. Pathogen-inactivating agent according to one or more of the previous claims, characterized in that the pathogen-inactivating agent contains riboflavin with a covalently bound iron EDTA complex, whereby the complex can be bound to riboflavin via a spacer.

21. Pathogen-inactivating agent according to claims 1 and 2, characterized in that the pathogens are viruses, bacteria, protozoans, and/or fungi carrying nucleotide.

22. Use of a pathogen-inactivating agent according to the claims 1 to 21, characterized in that the inactivation occurs by means of attachment to nucleic acids or pathogens, and by the destruction of the nucleic acids at this location.

23. Use according to claim 22, characterized in that the inactivation is started by means of the addition of an agent, preferably a reducing agent.

24. Use according to claim 23, characterized in that the reducing agent is ascorbic acid, or one of its salts.

25. Use according to claims 22 to 24, characterized in that the inactivation occurs in liquid.

26. Use according to claims 22 to 25, characterized in that the liquids are physiological solutions.

27. Use according to claim 26, characterized in that the physiological solutions are blood, or blood fractions.

28. Use according to claims 22 to 27, characterized in that the agent for the inactivation of pathogens or its fractions is removed after successful inactivation.

29. Use according to claims 22 to 27, characterized in that the agent for starting the reaction is removed after successful inactivation.
